(19) United States
(12) Patent Application Publication
(10) Pub. No.: US 2005/0079174 A1
(43) Pub. Date: Apr. 14, 2005

(54) Methods and compositions for immunotherapy of B cell involvement in promotion of a disease condition comprising multiple sclerosis

(75) Inventors: Emilio Barbera-Guillem, Powell, OH (US); M. Bud Nelson, Worthington, OH (US)

Correspondence Address: BENESCH, FRIEDLANDER, COPLAN & ARONOFF LLP;ATTN: IP DEPARTMENT DOCKET CLERK, 2300 BP TOWER, 200 PUBLIC SQUARE, CLEVELAND, OH, 44114, US

(73) Assignee: BioCrystal, Ltd., Westerville, OH (US)

(21) Appl. No.: 10/626,213

(22) Filed: Jul. 24, 2003

Related U.S. Application Data
(63) Continuation of application No. 09/643,595, filed on Aug. 22, 2000, now abandoned.
(60) Provisional application No. 60/152,498, filed on Sep. 2, 1999.
(60) Provisional application No. 60/150,256, filed on Aug. 23, 1999.

(51) Int. Cl. A61K 39/395

(52) U.S. Cl. 424/144.1

(57) ABSTRACT

Methods are provided for reducing a pro-multiple sclerosis immune response by administering to an individual a composition comprising an affinity ligand which binds to B cell determinant, and which is administered in an amount effective to reduce B cells.

### CROSS REFERENCE TO RELATED APPLICATIONS

This non-provisional application is a continuation of application Ser. No. 09/643,595, filed Aug. 22, 2000, which is a non-provisional application based on earlier co-pending provisional application Ser. No. 60/150,256, filed Aug. 23, 1999, now abandoned, and 60/152,498, filed Sep. 20, 1999, now abandoned, which are herein incorporated by reference.

### FIELD OF THE INVENTION

The present invention is related to novel methods for immunotherapy of an immune response that contributes to the pathological processes of a disease condition comprising multiple sclerosis in humans. More particularly, the present invention is related to the methods for treating B cells which may be involved in promotion of a chronic inflammatory process involving central nervous system tissue.

### BACKGROUND OF THE INVENTION

Multiple sclerosis (“MS”) is a chronic inflammatory disease of the central nervous system. The characteristic pathological feature, and still used as the primary basis for diagnosis of MS, is demyelination of the myelin sheath of neurons in the central nervous system. MS affects 250,000 to 350,000 in the United States, and approximately 1 million people worldwide. Typically, MS begins as a relapsing-remitting disease (RRMS) with periodic episodes of associated symptoms (e.g. various forms of neuritis). Often RRMS eventually changes to a progressive course of disease, secondary progressive MS (SPMS), characterized by more CNS tissue damage which results in more debilitating symptoms. However, in 10 to 20% of individuals, the disease initially develops in a progressive form known as primary progressive MS (PPMS).

There is no clear understanding of the immunopathogenic processes associated with MS; and, to date, their lacks evidence of a unique immunologic abnormality in individuals with MS (Whitaker, 1998, N. Engl. J. Med. 339:339-340; Rudick et al., 1997, N. Engl. J. Med. 337:1604-1611). Because of the incomplete understanding of the pathogenesis of MS, therapeutic advances have been slow to emerge. The myelin sheath and oligodendrocytes are believed to be main targets of autoreactive T cells which, when activated and reach the central nervous system, are thought to secrete proinflammatory cytokines. These cytokines are believed to induce astrocytes and leukocytes (including by activating microglia and macrophages) to secrete enzymes which damage myelin, and result in inflammation, demyelination, and axonal damage in the central nervous system characteristic in MS.

Thus, studies have implicated a cell mediated immune response, T cells recognizing epitopes of myelin basic protein (MBP), in the pathogenesis of MS. However, whether the principal effector cells are T cells, or macrophages, or both T cells and macrophages, is unknown. As shown in Table 1, currently therapies against MS are either directed to T cells, or are directed to suppressing the inflammation (e.g., by suppressing macrophages or microglial cells) that accompanies demyelination.

TABLE 1

| Drug | Action |
|---|---|
| Betaseron | reduces T cells & gamma interferon |
| beta 1-Alpha | reduces # of circulating T cells |
| Cladribine | reduces # of circulating T cells |
| Copolymer 1 | acts as a decoy for T cells |
| Methotrexate | reduces # of circulating T cells |
| Myloral | desensitizes T cells to myelin |
| beta interferon | suppresses microglial cells |
| methylprednisolone | suppresses macrophages |
| chlorambucil | suppresses inflammation |
| cyclophosphamide | suppresses inflammation |

Whether the humoral immune response is involved in the chronic inflammatory process underlying the pathology of MS is unproven. For example, anti-ganglioside antibodies have been reported to be elevated in MS patients (Acarin et al, 1996, Acta Neurol. Scand., 93:99-103; Sadatipour et al., 1998, Ann. Neurol., 44:980-983). However, it is unknown whether such antibodies are involved in axonal damage or are a consequence of axonal damage. Known pathology involving B cells is limited to such diseases as B cell lymphoma. There are numerous approaches and successes in treating B cell lymphoma (cancer cells of B cell origin). Such treatments include administration of immunologically active anti-CD20 antibodies to B cell lymphoma patients (see, e.g., U.S. Pat. No. 5,776,456); administration of an immunoconjugate comprising mAb Lym-1 coupled to ricin toxin A chain (see, e.g., U.S. Pat. No. 4,724,213); administration of an immunoconjugate comprising mAb LL2 (anti-CD22) coupled to chemotherapeutic agent (see, e.g., U.S. Pat. No. 5,789,554); and administration of an mAb alone, or an immunoconjugate comprising anti-CD19 mAb coupled to a chemotherapeutic agent (see, e.g., Hekman et al., 1991, Cancer Immunol. Immunother, 32:364-372; Cancer Research Weekly, Jun. 20, 1994, p.21; Cancer Research Weekly, Apr. 15, 1991, p.26).

We have discovered that certain soluble antigens, shed from central nervous system (CNS) tissue damaged by an inflammatory process, are capable of inducing an immune response which promotes one or more of the development, relapse, or progression of MS (“promotion of MS”). We have demonstrated that a similar phenomenon promotes a chronic inflammatory process which enables progression of solid, nonlymphoid tumors (see, e.g., Barbera-Guillem et al., 2000, Cancer Immunol, Immunother, 48:541.).

Therefore, a need exists for methods which may be used to therapeutically treat a disease condition selected from the group consisting of MS and a pro-MS immune response, and a pro-MS immune response, by treating B cells in an individual; particularly in an individual who has the disease condition. Further, there is a need to begin disease-modifying therapy early, before the development of irreversible tissue damage and resultant permanent disability.

### SUMMARY OF THE INVENTION

Accordingly, it is a primary object of the present invention to provide methods for treating B cells to inhibit their involvement in the promotion of MS.

It is another object of the present invention to provide methods for reducing a pro-MS immune response, wherein the treatment is directed to an individual's immune cells, wherein the immune cells are B lymphocytes, a subpopulation of which may be involved in the promotion of MS.

It is a further object of the present invention to provide methods for treating an individual with a composition for depleting B cells, including to a subpopulation of B cells which are activated by shed antigen released from CNS tissue damaged by an inflammatory process, which B cells may also be found circulating in body fluids selected from the group consisting of peripheral blood, cerebrospinal fluid, and a combination thereof (as disclosed in co-pending U.S. Ser. No. 60/150,256), wherein the depletion of B cells reduces inflammation which causes clinical manifestations associated with progressive MS.

It is also a further object of the present invention to provide methods for reducing a pro-MS immune response as an adjuvant regime in an individual who is apparent remission of MS, but whom still has the altered B cell subpopulations comprising a pro-MS immune response, and whom is therefore at risk for a relapse of MS.

The foregoing objects are based on a discovery of a novel mechanism in which shed antigen, particularly produced and shed by CNS tissue affected by an inflammatory process, is secreted and then induces (activates) a subpopulation of B cells, in a humoral immune response, to proliferate and differentiate into plasma cells which produce of anti-shed antigen antibody. Anti-shed antigen antibody can act indirectly and/or directly (via formation of complexes) to exacerbate the ongoing inflammatory process, thereby promoting progression of MS. The objects of the invention are also achieved by providing methods for depleting B cells, one or more subpopulations of which may be involved in promotion of MS and/or a pro-MS immune response. In one embodiment, administered to an individual is a composition in an amount effective to deplete B cells that may be present infiltrating CNS tissue involved in an inflammatory process; and/or (b) circulating in body fluids, such as peripheral blood and CSF; in an amount effective to deplete B cells. In another embodiment, administered to an individual is a therapeutically effective amount of one or more agents for treating MS (see, e.g., Table 1) in combination or conjunction with the composition for depleting B cells. In these embodiments, the function of the compositions of the present invention is to cause B cell depletion and reduce the exacerbation of inflammation, wherein B cell depletion may include one or more of: blocking of B cell function; functional inactivation of B cells; cytolysis of B cells; inhibiting the proliferation of B cells; inhibiting the production of antibody or inhibiting differentiation of shed antigen-specific B cells; and to cause inactivation or cytolysis of B cells which have been primed or activated by shed antigen. The compositions contact and bind to one or more determinants on B cells and may result in (cause and/or enable) B cell depletion, thereby immunomodulating the immune system to inhibit the MS-promoting function of B cells involved in a pro-MS immune response. Inhibiting the exacerbation of inflammation by depleting B cells involved in a pro-MS immune response may also inhibit a process selected from the group consisting of further development of MS, progression of MS, relapse of MS, and a combination thereof.

The above and other objects, features, and advantages of the present invention will be apparent in the following Detailed Description of the Invention when read in conjunction with the accompanying drawings.

### BRIEF DESCRIPTION OF THE DRAWINGS

FIG. 1 is a graph showing the effect of depletion on the number of CD19+ cells (B2 cells) of individuals over a treatment period with a composition in an amount effective to deplete B cells.

FIG. 2 is a graph showing the effect of depletion on the number of CD19+ sTn+ cells (B2 sTn+ cells) of individuals over a treatment period with a composition in an amount effective to deplete B cells.

FIG. 3 is a graph showing the effect of depletion on the number of CD19+ CD21+ sTn+ cells (sTn+ memory/mature B cells) of individuals over a treatment period with a composition in an amount effective to deplete B cells.

### DETAILED DESCRIPTION OF THE PREFERRED EMBODIMENTS

The terms “deplete” and “depletion” are used herein in reference to B cells, and for purposes of the specification and claims, to mean one or more of: blocking of B cell function; functional inactivation of B cells; cytolysis of B cells; inhibiting the proliferation of B cells; inhibiting the differentiation of B cells to plasma cells; causing a B cell dysfunction which results in a therapeutic benefit; inhibiting production of anti-shed antigen antibody; reduction in the number of B cells; inactivation of B cells which have been primed or activated by shed antigen; blocking of one or more functions of B cells which have been primed or activated by shed antigen; cytolysis of B cells which have been primed or activated by shed antigen; and reduction in the number of B cells which have been primed or activated by shed antigen. B cell depletion may be a result of one or more mechanisms including, but not limited to, clonal inactivation, apoptosis, antibody-dependent cellular cytotoxicity, complement-mediated cytotoxicity, and a signal pathway mediated inactivation, dysfunction, or cell death.

The term “composition” is used herein, for purposes of the specification and claims, to mean a composition (a) comprised of at least one affinity ligand which selectively (preferentially) binds to at least one determinant present on nonmalignant B cells (e.g., mature B cells and memory B cells); and (b) whereupon when administered in an effect amount to deplete B cells, upon contact and binding to such B cells, directly or indirectly results in (causes and/or enables) B cell depletion, particularly of shed antigen-specific B cells that may be involved in one or more of a pro-MS immune response, and promotion of progression of MS. Treatment with the composition may result in a beneficial function. Such a beneficial function may include, but is not limited to, one or more of: reduction of the pro-MS immune response by one or more processes selected from the group consisting of inhibiting the proliferation of B cells which may be involved, or may be recruited to be involved, in a pro-MS immune response, inhibiting production of the anti-shed antigen antibody (e.g., by reducing the number of B cells that can be induced to differentiate into anti-shed antigen antibody-secreting plasma cells), reducing the relative number (e.g., causing or enabling cytolysis) of B cells which have been primed or activated by shed antigen, and a combination thereof. By reducing a pro-MS immune response, the beneficial function may also comprise a reduction in (by inhibiting exacerbation of) the inflammatory process underlying clinical manifestations of MS. As an illustrative, but non-limiting, example, an anti-CD20 mAb, or an anti-Lym-1 mAb, or an anti-CD19 mAb, or an anti-CD21 mAb, may selectively bind to B cells (via CD20, Lym-1, CD19, and CD21, respectively) and facilitate or result in B cell depletion. A bi-specific antibody mAb, anti CD3-CD19 mAb, may bind to T cells (via CD3) and B cells (CD19) to mediate T cell-B cell interactions that may facilitate B cell depletion. Thus, in one embodiment, the present invention provides for the use of at least one affinity ligand in the manufacture of a pharmaceutical composition comprising a composition for a procedure for treating nonmalignant B cell involvement in one or more of a pro-MS immune response, and promotion of progression MS; wherein the at least one affinity ligand selectively contacts and binds to at least one determinant present on B cells selected from the group consisting of mature B cells and memory B cells, B1 cells, B cell subpopulations of altered amount in MS (see, e.g., Table 2), and a combination thereof; whereupon contact and binding to such B cells, the composition results in B cell depletion; and wherein the procedure comprises administering to an individual the composition in an amount effective to deplete B cells. Administering the composition to an individual may be by a mode which includes, but is not limited to, introducing the composition by a means to access the individual's CNS tissue to be treated (e.g., regional or “site-directed” treatment), introducing the composition into peripheral blood, and a combination thereof. The composition may further comprise at least one additional component selected from the group consisting of one or more chemotherapeutic agents, an anti-inflammatory agent, a cytolytic agent, a pharmaceutically acceptable carrier, and a combination thereof (see, e.g., Table 1). In one embodiment, the additional component is administered as a separate (nonconjugated) component of the composition. In another embodiment, the additional component may be coupled to the affinity ligand; wherein the affinity ligand serves to selectively bind the B cells, thereby bringing the additional component in contact with or in functional proximity of B cells that may be involved in the promotion of progression of MS. A cytolytic agent is an agent that, by interacting directly with such B cells, causes B cell cytotoxicity. Such cytolytic agents may include, but are not limited to, a therapeutically effective amount of an agent selected from one or more of the group consisting of a toxin, a drug, an enzyme, a cytokine, a radionuclides, a photodynamic agent, and a molecule which induce apoptosis (e.g., Fas ligand). A toxin may be selected from a therapeutically effective amount of one or more of the group consisting of ricin A chain, mutant Pseudomonas exotoxins, diphtheria toxoid, streptonigrin, boamycin, saporin, gelonin, and pokeweed antiviral protein. A drug may include a therapeutically effective amount of a cytotoxic drug selected from one or more of the group consisting of fludarabine, chlorambucil, danazol, daunorubicin, doxorubicin (e.g., in liposomes), cisplatin, bleomycin, melphalan, mitomycin-C, and methotrexate. Due to the sensitivity of B cells to radiation, radionuclides may include, but are not limited to, one or more radiometals such as yttrium which emits a high energy beta particle, and I¹²⁵ that emits Auger electrons, that may be absorbed by adjacent B cells. A photodynamic agent may include a therapeutically effective amount of a porphyrin or their derivatives. In addition to the anti-inflammatory agents listed in Table 1, other anti-inflammatory agents are known to those skilled in the art to include COX-2 inhibitors (e.g., rofecxib, and celecoxib), aspirin, ibuprofen, naproxen, and the like. The methods for coupling ligands or targeting molecules with therapeutic agents are well known to those skilled in the art (See, for example, conjugates as reviewed by Ghetie et al., 1994, Pharmacol. Ther. 63:209-34; U.S. Pat. No. 5,789,554, the disclosure of which is herein incorporated by reference). Often such methods utilize one of several available heterobifunctional reagents used for coupling or linking molecules. Also, the additional component may comprise a pharmaceutically acceptable carriers for facilitating infusion into the bloodstream, CSF, or regional access to the CNS tissue affected by an inflammatory process. Pharmaceutically acceptable carriers are known to those skilled in the art to include buffered saline solutions, buffered carbohydrate solutions, liposomes (Phillips et al., 1994, J. Immunother. Emphasis Tumor Immunol. 15:185-93), sterile water, and the like.

The term “determinant” with reference to B cells, is used herein, for purposes of the specification and claims, to mean a molecule which is preferentially expressed by B cells, or one or more subpopulations thereof which include memory B cells and mature B cells, and one or more B cell subpopulations of altered amounts in MS (see, e.g., Table 2); wherein the molecule is involved in and responsible for selective binding to an affinity ligand having binding specificity and avidity for the determinant. Cell-associated determinants may include, but are not limited to, molecules, receptors, components, or surface immunoglobulin, present on the surface of the cell membrane. “Preferentially expressed” is used herein to mean that the cell-associated determinant is expressed on a substantial number (approximately 40% or greater) of the B cells, or of a subpopulation thereof, which are targeted by the composition. In a preferred embodiment, the determinant is primarily expressed on B cells, with little or no expression of the determinant (as relative to the number of cells expressing the determinant or to the level of expression as compared to B cells) by other subpopulations of immune cells (with the possible exception of dendritic cells; e.g., CD21) contained within a region to which the composition is intended to be targeted. In a preferred embodiment, the determinant is selected from the group consisting of CD19, CD20 (see, e.g., U.S. Pat. No. 5,776,456, the disclosure of which is herein incorporated by reference), CD21, CD22 (see, e.g., LL2, U.S. Pat. No. 5,789,554, the disclosure of which is herein incorporated by reference; Erickson et al., 1996, Int. Immunol. 8:1121-9), Lym-1 (see, e.g., U.S. Pat. No. 5,789,554, the disclosure of which is herein incorporated by reference), CDIM (see, e.g., U.S. Pat. No. 5,593,676, the disclosure of which is herein incorporated by reference), slg having binding specificity for shed antigen, and a combination thereof.

The term “B cells” is used herein, for purposes of the specification and claims, and in reference to treating a disease condition selected from the group consisting of MS and a pro-MS immune response, and a pro-MS immune response, to mean mammalian (and preferably human) nonmalignant B cells. As known to those skilled in the art, malignant B cells refers to cancer cells of B cell origin, such as B cell lymphomas, and B cell leukemias. Thus, the term “B cells”, as used herein in reference to treating B cells and in treating the disease condition, specifically excludes B cell lymphomas, B cell leukemias, and cancer cells of B cell origin. In that regard, nonmalignant B cells are inclusive of one or more of memory B cells and mature B cells; and B cell subpopulations of altered amounts in MS (e.g., see Table 2) which are involved in one or more of a pro-MS immune response, and promotion of progression of MS, as will be more apparent from the following embodiments.

The term “affinity ligand” is used herein, for purposes of the specification and claims, to mean a molecule which has binding specificity and avidity for a determinant associated with B cells that may be present in the area of CNS tissue which is affected by a local inflammatory process (and which produces shed antigen), and/or that may be circulating in body fluids such as peripheral blood. In general, affinity ligands are known to those skilled in the art to include, but are not limited to, lectins (or fragments or derivatives thereof which retain specific binding activity), monoclonal antibodies (“mAb”, including chimeric or genetically modified monoclonal antibodies which may be preferable for administration to humans), peptides, and aptamers. The term “monoclonal antibody” is also used herein, for purposes of the specification and claims, to include immunoreactive fragments or immunoreactive derivatives (e.g., peptides) derived from a mAb molecule, which retain all or a portion of the binding function of the whole mAb molecule. Such immunoreactive fragments or immunoreactive derivatives are known to those skilled in the art to include F(ab¹)₂, Fab¹, Fab, Fv, scFV, Fd¹, Fd, and the like. Methods for producing the various fragments from mAbs are well known in the art (see, e.g., Plückthum, 1992, Immunol. Rev. 130:152-188). For example, F(ab¹)₂ can be produced by pepsin digestion of the monoclonal antibody, and Fab¹ may be produced by reducing the disulfide bridges of F(ab¹)₂ fragments. Fab fragments can be produced by papain digestion of the monoclonal antibody, whereas Fv can be prepared according to methods described in U.S. Pat. No. 4,642,334. Single chain derivatives can be produced as described in U.S. Pat. No. 4,946,778. The construction of chimeric antibodies is now a straightforward procedure (Adair, 1992, Immunological Reviews 130: 5-40,) in which the chimeric antibody is made by joining the murine variable region to a human constant region. Additionally, “humanized” antibodies may be made by joining the hypervariable regions of the murine monoclonal antibody to a constant region and portions of variable region (light chain and heavy chain) sequences of human immunoglobulins using one of several techniques known in the art (Adair, 1992, supra; Singer et al., 1993, J. Immunol. 150:2844-2857). Methods for making a chimeric nonhuman/human mAb in general, and a chimeric anti-CD20 mAb in particular, are described in detail in U.S. Pat. No. 5,736,137. The chimeric anti-CD20 antibody described in U.S. Pat. No. 5,736,137 has been reported to be therapeutically active on its own; e.g., does not require coupling to a toxin or radioisotope to induce cytolysis of targeted B cells. Likewise, chimeric anti-CD22 antibody has been previously described in U.S. Pat. No. 5,789,554. Likewise, a cross-linking of a B cell by an anti-CDIM mAb has been reported to induce a cellular response ultimately resulting in cell death (U.S. Pat. No. 5,593,676). In a preferred embodiment, affinity ligands may include, but are not limited to, a mAb having binding specificity for one of CD19, CD20, CD21, CD22, CDIM, or Lym-1. Aptamers can be made against B cell determinants using methods described in U.S. Pat. No. 5,789,157 (the disclosure of which is herein incorporated by reference).

The term “pro-MS immune response” is used herein, for purposes of the specification and claims, to mean a humoral immune response induced against an epitope comprising a terminal alpha 2,6 linked sialic acid (e.g., comprising sialyl Tn or sTn which comprises a terminal sialic acid alpha 2,6 linked to Ga1NAc; or alternatively, to Ga1) of a shed antigen (glycomolecule), resulting in production of IgG antibody against the epitope (“anti-α (2,6) NeuAc Ab”), and complexes comprised of the shed antigen comprising the epitope complexed to anti-α (2,6) NeuAc Ab; wherein the shed antigen is released or produced particularly in relation to CNS tissue damage characteristic of MS during the a chronic inflammatory disease process characteristic of inflammatory forms of MS (e.g., secondary progressive MS). In a preferred embodiment, the resultant complexes bind to and induce Fc receptor-expressing cells (e.g., one or more cell types selected from the group consisting of granulocytes, macrophages, microglia, activated mast cells, astrocytes, oligodendrocytes) which results in the release of inflammatory mediators (e.g., cytokines and/or tissue degradative enzymes) which may exacerbate the existing inflammatory process and thereby promote (contribute to) CNS tissue damage characteristic of MS (e.g., demyelination and plaques characteristic of MS). A similar immune response, a pro-tumor immune response, and it's ability to promote inflammation and tissue degradation has been described in co-pending U.S. application Ser. No. 09/435,289 (the disclosure of which is herein incorporated by reference). In a preferred embodiment, the anti-α (2,6) NeuAc Ab is induced by a shed antigen comprising glycolipid; and in a more preferred embodiment, glycolipid selected from one or more of the alpha series of gangliosides (e.g., GD1α, GT1aα, GQ1bα, derivatives thereof which contain one or more additional terminal sialic acids alpha 2,6 linked to Ga1NAc, and a combination thereof). Serological markers for a pro-MS immune response have been described in detail in co-pending U.S. applications Ser. Nos. 60/151,999 and 60/207,577, the disclosures of which are herein incorporated by reference.

The term “individual” is used herein, for purposes of the specification and claims, to mean a mammal; and preferably a human. An individual who is at risk of developing, or has developed, a pro-MS immune response may include an individual having a form of MS (e.g., secondary progressive form, or relapsing, remitting form); or an individual who is in apparent remission of MS, but whom harbors cellular markers characteristic of a pro-MS immune response (see Table 2) and therefore inherently carries a risk of relapse or recurrence. The method and compositions according to the present invention are intended for use to deplete B cells localized in inflammatory infiltrates in the region of CNS tissue damaged or affected by an inflammatory process, and/or circulating in body fluids selected from the group consisting of peripheral blood, CSF, and a combination thereof, in individuals at risk for developing, or who have developed, a pro-MS immune response.

The present invention relates to a discovery that in a pro-MS immune response, a significant number of activated B cells (memory B cells and mature B cells) may be retained locally as cells which infiltrate CNS tissue damaged or affected by an inflammatory process, and/or may be cells circulating in body fluids selected from the group consisting of peripheral blood, CSF, and a combination thereof. According to one embodiment of a method for treating B cell involvement in promotion of progression MS (also described herein as a method for reducing a pro-MS immune response) according to the present invention, administered to an individual is a composition in an amount effective to deplete B cells. The composition may be administered by a mode which facilitates infusion into an access which is regional to the CNS tissue damaged or affected by an inflammatory response (e.g., by a mode to facilitate delivery of the composition to demyelinating CNS tissue or plaques characteristic of MS), thereby delivering the composition in a site-directed manner. The composition may be administered by itself, or as part of a regimen of chemotherapeutic agents. In another embodiment of a method for reducing a pro-MS immune response according to the present invention, administered to an individual is the composition by a mode which facilitates infusion into peripheral blood (e.g., intravenously) and/or a vascular access (including lymphatics) of the individual's CNS tissue, wherein the composition is administered in an amount effective to deplete B cells. The composition may be administered by itself, or as part of a regimen of chemotherapeutic agents.

For purposes of the description, the methods and compositions of the present invention will be illustrated in the following examples.

### EXAMPLE 1

This embodiment provides evidence of the B cell involvement in a disease condition selected from the group consisting of MS and a pro-MS immune response, and a pro-MS immune response. We have discovered that there may exist altered phenotype in one or more B cell subpopulations, wherein the phenotype is altered because the relative amounts of these one or more B cell subpopulations that differ in individuals having disease condition selected from the group consisting of MS and a pro-MS immune response, as compared to those values in healthy controls or in individuals having inflammatory diseases other than MS. For detecting the alterations in the one or more B cell subpopulations in a clinical sample, mononuclear cells were isolated from the clinical sample using a density gradient medium and by density gradient centrifugation. Aliquots, each of approximately 1 million cells, were treated in one of several different ways.

Preferred Staining Combinations

Memory B Cells and sTn+ Memory B Cells

In determining a lymphocyte subpopulation comprising memory B cells, an aliquot of cells was double-stained using anti-CD19 antibody (IgG1 mAb) labeled with Pe-Cy5 (phycoerythrin-Cy5), and an anti-CD21 antibody (IgG1 mAb) labeled with FITC (fluorescein isothiocyanate), wherein the final dilution of each mAb was 1:10. The mixture was incubated for 30 minutes in the dark at 4° C., and then the mixture was centrifuged at 1500 rpm for 5 minutes. The supernatant was removed and a wash solution (e.g., 150 μl of a physiologically acceptable solution) was used to suspend the cell pellet, and then the mixture was centrifuged (a wash step). The wash step may be repeated one or more times. The cell pellet from the final wash was then taken up in a physiologically acceptable solution in a sufficient volume for flow cytometric analysis (e.g., 200-250 μl). For detecting sTn+ memory B cells, an aliquot of cells was triple-stained wherein the cells were first mixed and incubated with anti-sTn antibody (IgG1 mAb), and then washed; followed by mixing and incubating with a secondary rabbit anti-mouse IgG antibody labeled with Pe (phycoerythrin), and then washed; followed by a double-staining with anti-CD9 antibody labeled with Pe-Cy5, and an anti-CD21 antibody labeled with FITC, and then washed. Additionally, by gating on the appropriate parameters, a lymphocyte subpopulation comprising overall B cells (CD19+ cells) may be determined. By gating on the appropriate parameters, a lymphocyte subpopulation comprising sTn+ B cells (CD19+ sTn+ cells) may be determined.

B Cells and sTn+ B Cells

In determining a lymphocyte subpopulation comprising overall B cells, an aliquot of cells was double-stained using essentially the same protocol as summarized above, except that the antibodies mixed with the cells of the this aliquot were anti-CD19 antibody (IgG1 mAb) labeled with Pe-Cy5 (final dilution of each mAb was 1:10). Alternatively, for detecting sTn+ B cells, the cells were first mixed and incubated with anti-sTn antibody (IgGi mAb), and then washed; followed by mixing and incubating with a secondary rabbit anti-mouse IgG antibody labeled with Pe, and then washed; followed by staining with anti-CD19 antibody labeled with Pe-Cy5, and then washed. This alternative staining protocol, with the appropriate gating, allows for determination of both overall B cells (CD19+ cells) and sTn+ B cells (CD19+ sTn+ cells).

sTn+ B1 cells

In determining a lymphocyte subpopulation comprising sTn+ B1 cells, an aliquot of cells was triple-stained, wherein the cells were first mixed and incubated with anti-sTn antibody (IgG1 mAb), and then washed; followed by mixing and incubating with a secondary rabbit anti-mouse IgG antibody labeled with Pe, and then washed; followed by a double-staining with anti-CD19 antibody labeled with Pe-Cy5, and an anti-CD5 antibody labeled with FITC, and then washed. Staining and incubation times were similar to those described above. Additionally, by gating on the appropriate parameters, a lymphocyte subpopulation comprising overall B cells (CD19+ cells) may be determined. Also by gating on the appropriate parameters, overall B1 cells (CD19+ CD5+ cells) may be determined.

A number of commercially available flow cytometers can be used as the instrument on which is performed the method of the present invention. Desirably, the flow cytometer has a single laser source; and in a preferred embodiment, the single laser source is an argon laser tuned at 488 nanometers (nm). Additionally, the flow cytometer is operatively connected to appropriate operating software and data management systems. Using these methods, quantitated were the relative numbers of lymphocyte subpopulations comprising altered lymphocyte phenotype in clinical samples obtained from individuals having MS and/or a pro-MS immune response; and, as shown in Table 2, the results were compared to the relative numbers of the same types of lymphocyte subpopulations determined in clinical samples obtained from apparently healthy individuals (“Baseline control”) and determined in clinical samples obtained from individuals having solid, nonlymphoid tumors and/or a pro-tumor immune response. In this illustration, the clinical samples comprised peripheral blood obtained by venipuncture into blood collection tubes, wherein peripheral blood mononuclear cells were isolated and then analyzed; and the quantitations were performed using flow cytometric methods by the techniques disclosed herein. Light scatter was used as a parameter to gate on primarily lymphocytes based on the size, granularity and cell volume of lymphocytes. In addition to gating for light scatter, each sample undergoing the staining process was gated for respective fluorescence emission(s). For example, when memory B cells were quantitated by double-staining (e.g., for CD19 and CD21), the analysis was gated on those cells positive for CD19 expression as determined by detection of Pe-Cy5 fluorescent emission. CD19 positive lymphocytes were considered to represent the relative overall population of B cells in the clinical sample analyzed. CD19 positive lymphocytes were then gated for those cells also positive for CD21 expression as determined by detection of FITC fluorescent emission. Lymphocytes double stained for both CD19 and CD21 were considered to represent memory B cells. Such CD19+ CD21+ B cells were then expressed as a percentage of overall B cells by using the formula:
(the relative number of CD19+ CD21+ cells/relative number of CD19+ cells)×100.

A similar procedure was also used to detect and quantitate a sTn+ B cell subpopulation (e.g., CD19+ sTn+ B cells). Such CD19+ sTn+ B cells were expressed as a percentage of overall B cells by using the formula:
(the relative number of CD19+ sTn+ cells/relative number of CD19+ cells)×100.

A similar procedure was also used to detect and quantitate a sTn+ memory B cell subpopulation (e.g., CD19+ CD21+ sTn+ B cells). Such CD19+ CD21+ sTn+ B cells were expressed as a percentage of memory B cells by using the formula:
(the relative number of CD19+ CD21+ sTn+ cells/relative number of CD19+ CD21+ cells)×100.

A similar procedure was also used to detect and quantitate a B1 cell subpopulation (e.g., CD19+ CD5+ B cells). Such CD19+ CD5+ B cells were expressed as a percentage of overall B cells by using the formula:
(the relative number of CD19+ CD5+ cells/relative number of CD19+ cells)×100.

A similar procedure was also used to detect and quantitate an sTn+ B1 cell subpopulation (e.g., CD19+ CD5+ sTn+ cells). Such CD19+ CD5+ sTn+ B cells were expressed as a percentage of overall B1 cells by using the formula:
(the relative number of CD19+ CD5+ sTn+ cells/relative number of CD19+ CD5+ cells)×100.

In Table 2, for most B cell subpopulation determinations from apparently healthy individuals (Table 2, “Baseline control”) and for most determinations from individuals having solid, nonlymphoid tumors and/or a pro-tumor immune response (Table 2, “Tumor/PTIR”), the relative number is expressed as the mean± the standard error of the mean. SPMS/PMSIR represents lymphocyte subpopulation determinations from an individual with secondary progressive MS (SPMS) and with evidences of a pro-MS immune response (PMSIR) as determined by immunoassays for plasma (antished antigen antibody, shed antigen, and complexes of anti-shed antigen antibody+shed antigen).

TABLE 2

| lymphocyte | Baseline | | SPMS/ | RRMS/ |
|---|---|---|---|---|
| subpopulation | control | Tumor/PTIR | PMSIR | PMSIR |
| CD19+ — | 12.7 ± 3.6 | 2.7 ± 0.5 | 5.9 | 13.3 |
| CD19+ sTn+ | 1.3 ± 0.4 | 5.9 ± 1.5 | 18.2 | 1.6 |
| CD19+ CD21+ | 1.1 ± 0.7 | 48.8 ± 5.3 | 0.7 | 3.3 |
| CD19+CD21+sTn+ | 29.7 ± 5.0 | 16.1 ± 3.7 | 42.9 | 26.0 |
| CD19+ CD5+ | 1.8 | 2.4 | 2.2 | — |
| CD19+CD5+sTn+ | 0.3 | 29.0 | 17.3 | — |

Table 2 illustrates that one or more B cell subpopulations are altered in relative amount in an individual having MS and a pro-MS immune response. Note that there is a significant decrease in the relative number of overall B cells (CD19+ cells) in an individual having secondary progressive MS and pro-MS immune response (SPMS/PMSIR) as compared to values in apparently healthy individuals (Baseline control). There is a significant increase in the relative number of sTn+ B cells (CD19+ sTn+ cells) in an individual having SPMS/PMSIR as compared to values for the baseline control. While no significant difference was noted in an overall memory B cell population (CD19+ CD21+ cells), there is a significant increase in the relative number of sTn+ memory B cells (CD19+ CD21+ sTn+ cells) in an individual having SPMS/PMSIR as compared to values for the baseline control. While no significant difference was noted in an overall memory B1 cell population (CD19+ CD5+ cells), there is a significant increase in the relative number of sTn+ B1 cells (CD19+ CD5+ sTn+ cells) in an individual having SPMS/PMSIR as compared to values in the baseline control. Such alterations in the relative amounts of one or more B cell subpopulations was not observed in the less inflammatory form of MS comprising primary progressive MS.

### EXAMPLE 2

We have discovered that another indication of a pro-MS immune response is the presence in peripheral blood (e.g., as detected in serum or plasma), and additional evidence of B cell involvement in the promotion of MS, of complexes comprising shed antigen complexed to anti-shed antigen antibody. As an illustration, an enzyme-linked immunosorbent assay (ELISA) was performed. A polystyrene microtiter plate was coated with a film comprising an affinity ligand having binding specificity for a terminal 2,6 linked sialic acid. In this illustration, a mouse monoclonal antibody having such binding specificity was used to coat the wells at a concentration of 10 μg/ml. After blocking the wells with a blocking solution, the wells coated with the affinity ligand were then used to detect the presence of immune complexes in human plasma samples (diluted 1:50 in buffer) from:
(1) individuals with no apparent disease or pathology (“Control”);
(2) individuals having solid, nonlymphoid tumor (“Cancer”); and
(3) individuals having RRMS or SPMS, and a pro-MS immune response (“MS/PMSIR”).

The detector molecule comprised anti-human IgG labeled with peroxidase for complexed antibody of the IgG class, and anti-human IgM labeled with peroxidase for detecting complexed antibody of the IgM class. At the appropriate point in the immunoassay procedure, substrate comprising tetramethyl benzidine was added to determine peroxidase activity. The presence of immune complexes was detected by the yellow color (after addition of the sulfuric acid containing stopping reagent), and the color was quantitated at 450 nanometers using a plate reading spectrophotometer. Table 3 illustrates the amount of complexes (circulating complexes with complexed IgG, “CC IgG”; circulating complexes with complexed IgM, “CC IgM”) detected as measured by absorbance at 450 nm and expressed as the mean absorbance± standard error of the mean (Mean± s.e.m.).

TABLE 3

| plasma | CC IgM | CG IgC |
|---|---|---|
| source | Mean ± s.e.m. | Mean ± s.e.m. |
| Control | 0.37 ± 0.05 | 0.41 ± 0.04 |
| Cancer | 0.80 ± 0.33 | 0.68 ± 0.12 |
| MS/PMSIR | 0.11 ± 0.95 | 1.44 ± 0.19 |

As shown in Table 3, individuals having RRMS and a pro-MS immune response or SPMS and a pro-MS immune response (“MS/PMSIR”) have a significant increase in complexes comprised of shed antigen complexed to anti-shed antigen IgG antibody. Such a high concentration of this type of immune complex is an indication of a pro-MS immune response; and of B cell involvement in promotion of MS, wherein such complexes can activate immune effector cells to promote or exacerbate an inflammatory process and tissue degradation.

### EXAMPLE 3

The findings in an individual of one or more B cell subpopulations which are altered in individuals having a pro-MS immune response are indicative of the involvement of one or more subpopulation of B cells in promotion of progression of MS. In such an individual, the B cells may be found infiltrating CNS tissue undergoing demyelination due to an immune reaction and/or an inflammatory response, and/or may be found in body fluids selected from the group consisting of peripheral blood, CSF and a combination thereof. In a method of reducing a pro-MS immune response according to the present invention, administered to an individual is a composition in an amount effective for depleting B cells present in one or more of (a) CNS tissue undergoing demyelination due to an immune reaction and/or an inflammatory response; (b) in body fluids such as peripheral blood. The administered composition subsequently comes in contact with such B cells, and may result in one or more therapeutic functions which may include, but is not limited to, B cell depletion; inhibition of proliferation of B cells involved in a pro-MS immune response; inhibition of activation of B cells by shed antigen; inhibition of differentiation of shed antigen-activated B cells into plasma cells capable of secreting anti-shed antigen antibody; causing or enabling inactivation and/or cytolysis of memory B cells which may include a subpopulation of memory B cells that have been primed or activated by shed antigen; and causing or enabling inactivation and/or cytolysis of the one or more B cell subpopulations found in an altered amount (see, e.g., Table 2).

In another embodiment, a method for inhibiting the chronic inflammatory process associated with MS and a pro-MS immune response comprises administering the composition in an effective amount to deplete B cells, wherein the composition contacts and binds to one or more determinants on B cells to cause or enable B cell depletion, thereby inhibiting the involvement of B cells in contributing to the chronic inflammatory process which promotes a progression of MS.

The nerves of the central nervous system which undergo demyelination in MS are often proximal to one or more accesses (e.g., blood or lymphatic or spaces (subarachnoid space)) that feed or extend through the CNS tissue. Thus, to deplete B cells that infiltrate CNS tissues, administered is the composition in one or more accesses in a site-directed method of delivery, wherein the composition is administered in an amount effective to deplete B cells. In one example, it is known that some drugs (e.g., baclofen) are administered intrathecally by bolus injection or by infusions administered by a programmable pump implanted in the lumbar subarachnoid space. Using standard methods for site-directed delivery, as known to those skilled in the art, the composition may be infused into one or more accesses that directly supplies CNS tissue undergoing demyelination (“affected CNS tissue”). The delivered composition may then concentrate primarily in the affected CNS tissue; and more specifically to the B cells present, by the binding between the affinity ligand and its target determinant on the B cells. Site-directed delivery of the composition does not rule out that a portion of the composition may gain access to peripheral blood, and any benefit associated therewith. Likewise, infusion intravenously may result in the composition reaching B cells infiltrating the affected CNS tissue. This is due, in part, to the increased blood-brain barrier permeability that is often observed in individuals having early or late stage development of a chronic progressive form of MS, and as particularly localized in demyelinated plaques. The composition may be administered by itself, or in conjunction with as additional component as previously described herein in more detail. Also, the composition may further comprise a pharmaceutically acceptable carrier for facilitating infusion.

### EXAMPLE 4

In this example, illustrated is an embodiment of reducing a pro-MS immune response according to the present invention, wherein the composition is administered parenterally in an amount effective to deplete B cells. The term “parenterally” includes administration intravenously, intramuscularly, subcutaneously, rectally, vaginally, or intraperitoneally. The most preferred parenteral administration is intravenous administration. As will be apparent to one skilled in the art, an amount effective to deplete B cells, and whether repeated dosages may be warranted, will depend on such factors as the stage of development of the inflammatory process of MS and/or a pro-MS immune response, overall health of the individual to be treated, other treatments which the individual may be undergoing, and pharmacokinetic properties of the type of the composition being used. For example, for a composition comprising a chimeric anti-CD20 mAb, an effective dose may range from about 0.01 mg/kg of body weight to about 40 mg/kg of body weight. However, as apparent to one skilled in the art, and in the discretion of a medical practitioner, a treatment may be warranted with a dosage falling inside or outside of this illustrative range. In an illustration of parenteral administration, a composition comprising a chimeric anti-CD20 mAb may be administered by intravenous infusion in an effective amount to deplete B cells. The dosage or regimen (multiple doses) may be determined by monitoring one or more of changes in the status of MS disease, and measurable parameters of efficacy of the treatment. Various measurable parameters may include, but are not limited to, the counts or relative numbers of peripheral blood B lymphocyte subpopulations (e.g., one or more sTn+ B cell subpopulations, and other B cell subpopulations; see, e.g., Table 2), the CD4/CD8 ratio of peripheral blood lymphocytes, serum or plasma concentration of complexes comprising shed antigen complexed to anti-shed antigen IgG antibody, and imaging of the affected CNS tissue before and after treatment.

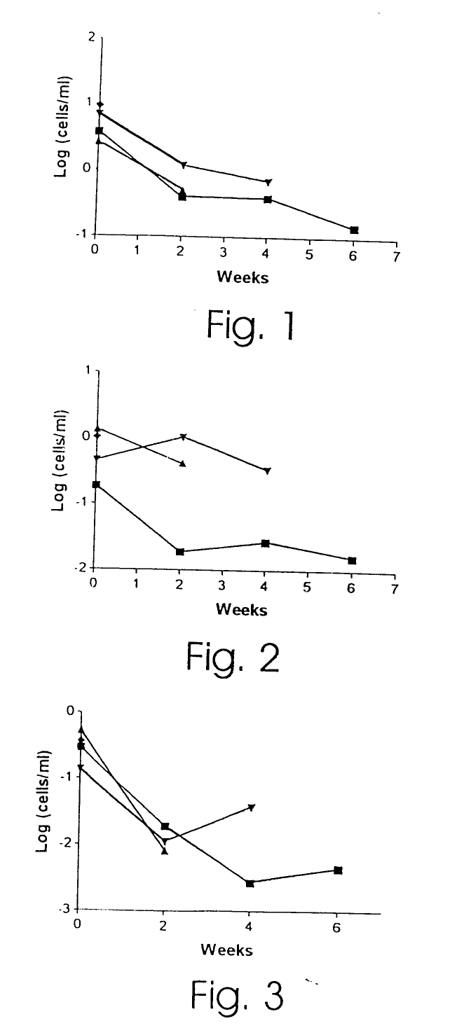

To illustrate the method of depleting B cells according to the present invention, administered to individuals having altered B cell subpopulations (see Table 2) was a composition comprising a chimeric anti-CD20 mAb administered by intravenous infusion in an amount effective to deplete B cells. Each individual received 200 mg of the composition, and then received two additional infusions approximately every four weeks. Thus, three complete infusions were administered: the initial treatment (week 0), one at week 4, and one at week 8. Generally, the first infusion was at an initial rate of about 50 mg/hour; however, additional infusions were administered at a faster rate which was dependent on how the individual tolerated infusion, the treating physician's judgment, drug manufacturer's instructions, and lack of side effects. As shown in FIGS. 1-3, treatment according to the present invention of 3 individuals (▪, ▴,▾) with the composition resulted in a depletion in overall B cells (CD19+ cells; FIG. 1), and also normalization of the amounts of B cells comprising B cell subpopulations altered in amount (FIG. 2, CD19+ sTn+ cells; FIG. 3, CD19+ CD21+ sTn+ cells) to within a range observed in apparently healthy individuals. Such an observed effect of the composition on depletion of B cells have resulted in a clinical benefit to treated individuals.

The foregoing description of the specific embodiments of the present invention have been described in detail for purposes of illustration. In view of the descriptions and illustrations, others skilled in the art can, by applying, current knowledge, readily modify and/or adapt the present invention for various applications without departing from the basic concept, and therefore such modifications and/or adaptations are intended to be within the meaning and scope of the appended claims.

1. A method for reducing a pro-multiple sclerosis immune response in an individual, wherein the pro-MS immune response comprises a humoral immune response induced against an epitope comprising terminal alpha 2,6 linked sialic acid on shed antigen, the method comprising administering to the individual a composition comprising an affinity ligand which selectively binds to a B cell determinant, wherein the B cell determinant is selected from the group consisting of CD19, CD20, CD21, CD22, Lym-1, and a determinant expressed only by B cells and not by immune cells other than B cells; wherein the B cells targeted by the method and by the composition are nonmalignant B cells, wherein the composition is administered in an amount effective to deplete B cells, and wherein the depletion of B cells results in reducing the pro-multiple sclerosis immune response induced against the epitope comprising terminal alpha 2,6 linked sialic acid.

2-17. (canceled)

18. The method according to claim 1, wherein the nonmalignant B cells are B cells selected from the group consisting of mature B cells, memory B cells, CD19⁺sTn⁺ B cells, CD19⁺CD21⁺sTn⁺ B cells, and CD19⁺CD5⁺sTn⁺ B cells, and a combination thereof.

19. The method according to claim 1, wherein the composition comprises a chimeric anti-CD20 monoclonal antibody.

20. The method according to claim 1, wherein the composition is administered parenterally, or in a site directed method in which the composition is delivered into an access that directly supplies central nervous tissue undergoing demyelination.

21. The method according to claim 1, wherein the composition further comprises an additional component selected from the group consisting of one or more chemotherapeutic agents, an anti-inflammatory agent, a cytolytic agent, a pharmaceutically acceptable carrier, and a combination thereof.

22. The method according to claim 1, wherein the shed antigen comprises a glycolipid comprising one or more epitopes comprising terminal alpha 2,6 linked sialic acid.

23. The method according to claim 22, wherein glycolipid comprises a ganglioside.

24. The method according to claim 1, wherein the composition comprises an antibody.

25. The method according to claim 1, wherein the composition is administered intravenously.

26. A site-directed method for reducing a pro-multiple sclerosis immune response in an individual, wherein the pro-multiple sclerosis immune response is a humoral immune response induced against an epitope comprising a terminal alpha 2,6 linked sialic acid on shed antigen, the method comprising administering to the individual a composition comprising an affinity ligand, which selectively binds to a B cell determinant, wherein the B cell determinant is selected from the group consisting of CD19, CD20, CD21, CD22, Lym-1, and a determinant expressed only by B cells and not by immune cells other than B cells; wherein B cells targeted by the method and by the composition are nonmalignant B cells, wherein the composition is delivered into an access that directly supplies central nervous tissue undergoing demyelination, wherein the composition is administered in an amount effective to deplete B cells, and wherein the depletion of B cells results in reducing the pro-multiple sclerosis immune response induced against the epitope comprising terminal alpha 2,6 linked sialic acid epitope.

27. The method according to claim 26, wherein the nonmalignant B cells are B cells selected from the group consisting of mature B cells, memory B cells, CD19⁺sTn⁺ B cells, CD19⁺CD21⁺sTn⁺ B cells, and CD19⁺CD5⁺sTn⁺ B cells, and a combination thereof.

28. The method according to claim 26, wherein the composition comprises a chimeric anti-CD20 monoclonal antibody.

29. The method according to claim 26, wherein the composition further comprises an additional component selected from the group consisting of one or more chemotherapeutic agents, an anti-inflammatory agent, a cytolytic agent, a pharmaceutically acceptable carrier, and a combination thereof.

30. The method according to claim 26, wherein the shed antigen comprises a glycolipid comprising one or more epitopes comprising terminal alpha 2,6 linked sialic acid.

31. The method according to claim 30, wherein glycolipid comprises a ganglioside.

32. The method according to claim 26, wherein the composition comprises an antibody.

33. A method for reducing a pro-multiple sclerosis immune response in an individual, wherein the pro-multiple sclerosis immune response is directed against an epitope comprising terminal alpha 2,6 linked sialic acid contained on shed antigen comprising a glycolipid, the method comprising administering to the individual a composition comprising a monoclonal antibody, wherein the monoclonal antibody binds to a B cell determinant selected from the group consisting of CD19, CD20, CD21, CD22, Lym-1, and a determinant expressed only by B cells and not by immune cells other than B cells; wherein B cells targeted by the method and by the composition are nonmalignant B cells, and wherein the composition is administered in an amount effective to deplete B cells such that said pro-MS immune response is reduced.

34. The method according to claim 33, wherein the nonmalignant B cells are B cells selected from the group consisting of mature B cells, memory B cells, CD19⁺sTn⁺ B cells, CD19⁺CD21⁺sTn⁺ B cells, and CD19⁺CD5⁺sTn⁺ B cells, and a combination thereof.

35. The method according to claim 33, wherein the monoclonal antibody comprises a chimeric anti-CD20 monoclonal antibody.

36. The method according to claim 33, wherein the composition further comprises an additional component selected from the group consisting of one or more chemotherapeutic agents, an anti-inflammatory agent, a cytolytic agent, a pharmaceutically acceptable carrier, and a combination thereof.

37. The method according to claim 33, wherein glycolipid comprises a ganglioside.

38. A method for treating inflammation associated with multiple sclerosis, wherein the inflammation is caused by a humoral immune response against a shed antigen comprising an epitope comprising a terminal alpha 2,6 linked sialic acid, the method comprising depleting B cells to inhibit said humoral immune response by administering an amount of a composition effective to deplete B cells and reduce said humoral immune response against the shed antigen, wherein the composition comprises an affinity ligand which binds to a B cell determinant selected from the group consisting of CD19, CD20, CD21, CD22, Lym-1, and a determinant expressed only by the B cells and not by immune cells other than B cells; and wherein B cells targeted by the method and by the composition are nonmalignant B cells.

39. The method according to claim 38, wherein the nonmalignant B cells are B cells selected from the group consisting of mature B cells, memory B cells, CD19⁺ sTn⁺ B cells, CD19⁺CD21⁺sTn⁺ B cells, and CD19⁺CD5⁺sTn⁺ B cells, or a combination thereof.

40. The method according to claim 38, wherein the composition comprises a chimeric anti-CD20 monoclonal antibody.

41. The method according to claim 38, wherein the composition further comprises an additional component selected from the group consisting of one or more chemotherapeutic agents, an anti-inflammatory agent, a cytolytic agent, a pharmaceutically acceptable carrier, and a combination thereof.

42. The method according to claim 38, wherein the composition comprises a monoclonal antibody.

43. The method according to claim 38, wherein the shed antigen comprises a glycolipid comprising one or more epitopes comprising terminal alpha 2,6 linked sialic acid.

44. The method according to claim 43, wherein glycolipid comprises a ganglioside.

45. A method for reducing a pro-multiple sclerosis immune response comprising administering to an individual an affinity ligand which selectively binds to a B cell determinant of a shed antigen-specific B cell, wherein the B cells are nonmalignant B cells.

46. The method according to claim 45, wherein the B cell determinant is selected from the group consisting of CD19, CD20, CD21, CD22 Lym-1 and a determinant expressed only by the B cells and not by immune cells other than B cells.

47. The method according to claim 45, wherein the nonmalignant B cells are B cells selected from the group consisting of mature B cells, memory B cells, CD19⁺ sTn⁺ B cells, CD19⁺CD21⁺sTn⁺ B cells, and CD19⁺CD5⁺sTn⁺ B cells, or a combination thereof.

48. The method according to claim 45, wherein the shed antigen-specific B cells have specificity for an epitope comprising terminal alpha 2, 6 linked sialic acid.
